(19) United States
(12) Patent Application Publication
(10) Pub. No.: US 2024/0382114 A1
(43) Pub. Date: Nov. 21, 2024

(54) PERSPIRATION ANALYSIS DEVICE AND METHOD

(71) Applicant: Nippon Telegraph and Telephone Corporation, Tokyo (JP)

(72) Inventor: Yuki Hashimoto, Tokyo (JP)

(21) Appl. No.: 18/692,197

(22) PCT Filed: Oct. 26, 2021
(86) PCT No.: PCT/JP2021/039419

§ 371 Date: Mar. 14, 2024

(51) Int. Cl. A61B 5/145; A61B 5/00

(52) U.S. Cl. CPC A61B 5/14517; A61B 5/14546; A61B 5/4266; A61B 5/6801; A61B 2560/0209

(57) ABSTRACT

A sweating analysis device includes a wearable sensor that outputs an electrical signal derived from an amount of sweat and an electrolyte concentration in the sweat, the sweat being secreted from a skin of a wearer, a sweat amount calculation unit that calculates the amount of sweat of the wearer, an electrolyte concentration calculation unit that calculates the electrolyte concentration in the sweat of the wearer, a power supply control unit that stops the supply of power to the wearable sensor, the sweat amount calculation unit and the electrolyte concentration calculation unit when a supply-of-power stop condition is satisfied, and restarts the supply of power when the supply-of-power stop period is ended, and a stop period calculation unit that calculates a supply-of-power stop period on the basis of the electrical signal obtained by the wearable sensor.

### CROSS-REFERENCE TO RELATED APPLICATIONS

This application is a national phase entry of PCT Application No. PCT/JP2021/039419, filed on Oct. 26, 2021, which application is hereby incorporated herein by reference.

### TECHNICAL FIELD

The present invention relates to a sweating analysis device and method for measuring an amount of sweat and an electrolyte concentration in sweat of a wearer by being worn on a human body.

### BACKGROUND

With global warming, the number of heat wave occurrences is increasing in most parts of the world. As the frequency of scorching heat increases, the incidence of heat stroke also increases. Also in Japan, the number of heat stroke carriers in recent years has maintained a high level since 2018, which is a social problem.

Heat stroke represents a state of multiple organ failure caused by an increase in metabolism due to a high-temperature environment or strenuous exercise. Generally, human body temperature is maintained at about 37° C. through a process of thermoregulation by the anterior hypothalamus. Several mechanisms associated with sweating (vaporization, radiation, convection, conduction, or the like) function to cool body surfaces. As body temperature increases, skin vasodilation due to active sympathetic activity increases a blood flow in the skin, and thus thermal sweating starts. Skin vasodilation causes a relative decrease in intravascular volume and causes heat syncope. The loss of salt and water due to sweat causes dehydration and salt depletion, with thermal fatigue and spasm. When salt and water are further lost, the thermoregulation function decreases, and subsequently the blood flow in internal organs decreases due to the outflow of blood from the central circulation to the skin and muscles, leading to organ failure, resulting in a so-called heat stroke.

As described above, water and salt in a human body play an important role in thermoregulation. In order to prevent dehydration symptoms that cause thermal fatigue, it is important to replenish appropriate amounts of water and salt. It is reported in many documents that a salinity concentration contained in sweat increases linearly with an increase in an amount of sweat. However, an increase rate of the salinity concentration varies depending on the sweating ability of a person and the salinity reabsorption ability in the sweat glands, and thus there are individual differences. It is also known that an individual's sweating ability and salinity reabsorption ability change under the influence of heat acclimation. Therefore, in order to monitor the loss of water and salt, it is considered effective to continuously monitor both an amount of sweat and a salinity concentration contained in sweat.

As the related art for realizing this monitoring, a wearable sensor for simultaneously measuring an amount of sweat and an electrolyte concentration has been proposed (see Patent Literature 1).

FIG. 8 is a sectional view of a wearable sensor disclosed in Patent Literature 1, and FIG. 9 is an enlarged view of FIG. 8. A wearable sensor 1 includes a base material 10 having a through-hole 11 serving as a liquid flow path and a recess 12 communicating with an end of the through-hole 11 on an outlet side, an electrode 14 disposed on a surface of the base material 10 in which the end of the through-hole 11 on an inlet side is open, a water absorbing structure 15 disposed on the surface of the base material 10 on the outlet side to be in contact with a liquid flowing out from an opening of the through-hole 11 on the outlet side to the recess 12, and a water absorbing electrode 16 disposed on a surface of the water absorbing structure 15 facing the base material 10 to face the opening of the through-hole 11 on the outlet side.

By using the wearable sensor 1 illustrated in FIGS. 8 and 9, an amount of sweat and an electrolyte concentration of a wearer of the wearable sensor 1 can be calculated on the basis of energization characteristics between the electrodes 14 and 16 due to sweat 102 flowing out from the through-hole 11 to the recess 12.

However, in the structure illustrated in FIGS. 8 and 9, when a sweat droplet 102a comes into contact with the electrode 16, a current flows between the electrodes 14 and 16, and when the droplet 102a disappears, a current does not flow. Thus, there is a problem that a current continues to flow during a period in which the droplet 102a exists between the electrodes 14 and 16, and power consumption increases.

### CITATION LIST

### Patent Literature

Patent Literature 1: WO 2021/038742 A

### SUMMARY

### Technical Problem

Embodiments of the present invention have been made to solve the above problems, and an object of embodiments of the present invention is to provide a sweating analysis device and a method capable of performing measurement of an amount of sweat and an electrolyte concentration in sweat while saving power.

### Solution to Problem

According to embodiments of the present invention, there is provided a sweating analysis device including a wearable sensor configured to output an electrical signal derived from an amount of sweat and an electrolyte concentration in the sweat, the sweat being secreted from a wearer's skin; a sweat amount calculation unit configured to calculate the amount of sweat of the wearer on the basis of the electrical signal obtained by the wearable sensor; an electrolyte concentration calculation unit configured to calculate the electrolyte concentration in the sweat of the wearer on the basis of the electrical signal obtained by the wearable sensor; a power supply unit configured to supply power to the wearable sensor, the sweat amount calculation unit, and the electrolyte concentration calculation unit; a power supply control unit configured to stop supplying the power from the power supply unit to the wearable sensor, the sweat amount calculation unit, and the electrolyte concentration calculation unit when a supply-of-power stop condition is satisfied, and restart supplying the power to the wearable sensor, the sweat amount calculation unit, and the electrolyte concentration calculation unit when a supply-of-power stop period is ended; and a stop period calculation unit configured to calculate the supply-of-power stop period on the basis of the electrical signal obtained by the wearable sensor.

In a configuration example of the sweating analysis device of embodiments of the present invention, electrical signal is a current that is changed by a droplet intermittently generated in the wearable sensor due to sweating of the wearer, and the power supply control unit determines that the supply-of-power stop condition is satisfied when a certain period of time has elapsed from a time point at which a change in the current starts to appear due to the droplet.

In a configuration example of the sweating analysis device of embodiments of the present invention, the electrical signal is a current that is changed by a droplet intermittently generated in the wearable sensor due to sweating of the wearer, and the power supply control unit determines that the supply-of-power stop condition is satisfied when a peak of the current occurs after supply of the power to the wearable sensor, the sweat amount calculation unit, and the electrolyte concentration calculation unit is started.

In a configuration example of the sweating analysis device of embodiments of the present invention, the electrical signal is a current that is changed by a droplet intermittently generated in the wearable sensor due to sweating of the wearer, and when t₁ is a time from a time point at which a change in the current starts appearing due to the droplet to a supply-of-power stop time point at which the supply-of-power stop condition is satisfied, tₘᵢₙ is a cycle of the peak occurring in the current and corresponding to a known maximum amount of sweat of the wearer, and t₂ is the supply-of-power stop period, the stop period calculation unit calculates the supply-of-power stop period t₂ to satisfy 0<t₂<tₘᵢₙ−t₁.

In a configuration example of the sweating analysis device of embodiments of the present invention, the electrical signal is a current that is changed by a droplet intermittently generated in the wearable sensor due to sweating of the wearer, and when t is a time from a time point at which a change in the current starts appearing due to the droplet to a supply-of-power stop time point at which the supply-of-power stop condition is satisfied, Q is the amount of sweat calculated by the sweat amount calculation unit, t is a cycle of a peak occurring in the current and corresponding to the amount of sweat Q, and t₂ is the supply-of-power stop period, the stop period calculation unit calculates the supply-of-power stop period t₂ to satisfy 0<t₂<t−t₁.

In a configuration example of the sweating analysis device of embodiments of the present invention, the electrical signal is a current that is changed by a droplet intermittently generated in the wearable sensor due to sweating of the wearer, and when t₁ is a time from a time point at which a change in the current starts appearing due to the droplet to a supply-of-power stop time point at which the supply-of-power stop condition is satisfied, Q is the amount of sweat calculated by the sweat amount calculation unit, Qₘₐₓ is a known maximum amount of sweat the wearer, tₘᵢₙ is a cycle of a peak occurring in the current and corresponding to the maximum amount of sweat Qₘₐₓ, and t₂ is the supply-of-power stop period, the stop period calculation unit calculates a cycle t* of the peak occurring in the current and corresponding to an amount of sweat Q+ΔQ and calculates the supply-of-power stop period t₂ to satisfy 0<t_{2<}t*−t₁ in a case where the sum Q+ΔQ of the amount of sweat Q and a predetermined value ΔQ is less than the maximum amount of sweat Qₘₐₓ, and calculates the supply-of-power stop period t₂ to satisfy 0<t_{2<}tₘᵢₙ−t₁ in a case where the sum Q+ΔQ of the amount of sweat Q and the predetermined value ΔQ is equal to or more than the maximum amount of sweat Qₘₐₓ.

In a configuration example of the sweating analysis device of embodiments of the present invention, the electrical signal is a current that is changed by a droplet intermittently generated in the wearable sensor due to sweating of the wearer, the sweat amount calculation unit calculates the amount of sweat of the wearer on the basis of a cycle of a peak of the current, and the electrolyte concentration calculation unit calculates the electrolyte concentration in the sweat of the wearer on the basis of a value of the peak of the current.

According to embodiments of the present invention, there is provided a sweating analysis method including a first step of detecting, by a wearable sensor, an electrical signal derived from an amount of sweat and an electrolyte concentration in the sweat, the sweat being secreted from a wearer's skin; a second step of calculating, by a sweat amount calculation unit, an amount of sweat of the wearer on the basis of an electrical signal obtained by the wearable sensor; a third step of calculating, by an electrolyte concentration calculation unit, an electrolyte concentration in sweat of the wearer on the basis of the electrical signal obtained by the wearable sensor; a fourth step of stopping supply of power from a power supply unit to the wearable sensor, the sweat amount calculation unit, and the electrolyte concentration calculation unit when a supply-of-power stop condition is satisfied; a fifth step of calculating a supply-of-power stop period on the basis of the electrical signal obtained by the wearable sensor; and a sixth step of restarting the supply of power to the wearable sensor, the sweat amount calculation unit, and the electrolyte concentration calculation unit when the supply-of-power stop period is ended.

### Advantageous Effects of Embodiments of Invention

According to embodiments of the present invention, by providing the power supply control unit and the stop period calculation unit, power is supplied to the wearable sensor, the sweat amount calculation unit, and the electrolyte concentration calculation unit only for the time required for calculating an amount of sweat and the electrolyte concentration during the period in which sweat exists between the electrodes of the wearable sensor, so that power consumption of the sweating analysis device can be reduced.

### BRIEF DESCRIPTION OF THE DRAWINGS

FIG. 1 is a diagram illustrating an example of a change in a value of a current flowing between electrodes measured by a conventional wearable sensor.

FIG. 2 is a block diagram illustrating a configuration of a sweating analysis device according to a first embodiment of the present invention.

FIG. 3 is a functional block diagram of an MCU of the sweating analysis device according to the first embodiment of the present invention.

FIG. 4 is a flowchart for describing an operation of the sweating analysis device according to the first embodiment of the present invention.

FIG. 5 is a sectional view of a wearable sensor of a sweating analysis device according to a second embodiment of the present invention.

FIG. 6 is an enlarged sectional view of FIG. 5.

FIG. 7 is a block diagram illustrating a configuration example of a computer that implements the sweating analysis device according to first and second embodiments of the present invention.

FIG. 8 is a sectional view of a conventional wearable sensor.

FIG. 9 is an enlarged sectional view of FIG. 8.

### DETAILED DESCRIPTION OF ILLUSTRATIVE EMBODIMENTS

### Principle of Embodiments of Invention

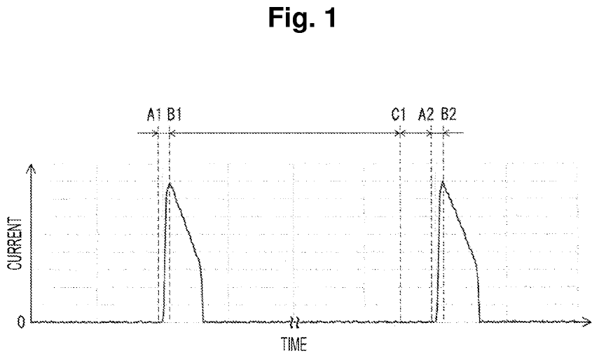

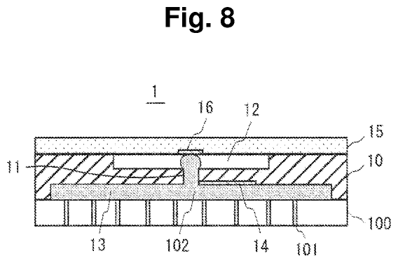

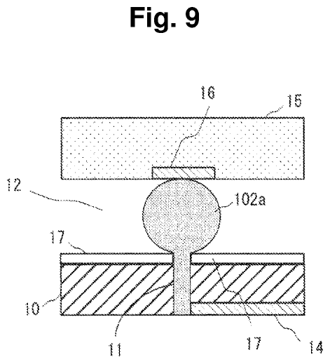

In order to describe the principle of embodiments of the present invention, an example of a change in a value of a current flowing between electrodes measured by the wearable sensor disclosed in Patent Literature 1 is illustrated in FIG. 1. As described above, in the structure illustrated in FIGS. 8 and 9, the current continues to flow during the period in which sweat droplets exist between the two electrodes, an energization cycle changes according to a variation in a flow rate of sweat flowing into the sensor, that is, an amount of sweat, and a peak current at the time of energization changes according to a variation in an electrolyte concentration.

For example, time points at which the energization between the two electrodes starts is A1, A2, . . . . In embodiments of the present invention, a time point B1 is set such that a time point at which a peak current occurs is included between the time point A1 and the time point B1, and the power supply is controlled such that the measurement by the sensor is performed only between the time points A1 and B1, and thereafter, the measurement by the sensor is not performed until a preset time point (C1 in FIG. 1). Thereafter, similarly, by performing power supply control by setting a period in which measurement is performed by the sensor and a period in which measurement is not performed by the sensor, measurement is performed by the sensor only for a time required for calculating an amount of sweat and an electrolyte concentration, and power consumption is reduced.

### First Embodiment

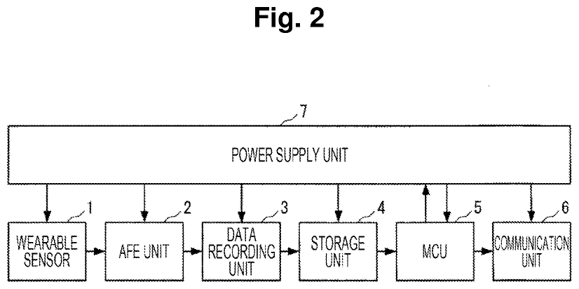

Hereinafter, H embodiments of the present invention will be described with reference to the drawings. FIG. 2 is a block diagram illustrating a configuration of a sweating analysis device according to a first embodiment of the present invention. The sweating analysis device includes a wearable sensor 1, an analog front end (AFE) unit 2, a data recording unit 3, a storage unit 4, a micro control unit (MCU) 5, a communication unit 6, and a power supply unit 7.

The wearable sensor 1 outputs an electrical signal (for example, a current with a waveform illustrated in FIG. 1) derived from an amount of sweat and an electrolyte concentration in the sweat, the sweat being secreted from a wearer's skin.

The AFE unit 2 includes an analog front end and amplifies a weak electrical signal output from the wearable sensor 1.

The data recording unit 3 includes an analog digital converter (ADC), converts the analog signal amplified by the AFE unit 2 into digital data at a predetermined sampling frequency, and stores the digital data in the storage unit 4.

The storage unit 4 stores the digital data output from the data recording unit 3. The storage unit 4 is realized by a nonvolatile memory typified by a flash memory, a volatile memory such as a dynamic random access memory (DRAM), or the like.

The MCU 5 is a circuit that performs signal processing of calculating an amount of sweat and an electrolyte concentration from the digital data stored in the storage unit 4, and power supply control.

The communication unit 6 includes a circuit that transmits the measurement result and the analysis result obtained by the MCU 5 to an external device (not illustrated) such as a smartphone in a wireless or wired manner. Examples of the wireless communication standard include Bluetooth (registered trademark) Low Energy (BLE). Examples of the wired communication standard include Ethernet (registered trademark).

The power supply unit 7 is a circuit that has a function of supplying power to the sweating analysis device.

Since the wearable sensor 1 in the present embodiment is the same as that disclosed in Patent Literature 1, a structure of the wearable sensor 1 will be briefly described with reference to FIGS. 8 and 9.

As described above, the wearable sensor 1 includes the base material 10, the electrodes 14 and 16, and the water absorbing structure 15.

As the base material 10, for example, there is one made of a hydrophilic glass material or resin material. The base material 10 may be obtained by subjecting the surface of a water-repellent material and the inner surface of the through-hole 11 to surface treatment for imparting hydrophilicity. A recess 12 having a recessed upper surface is formed on the upper surface of the base material 10 to communicate with the through-hole 11. On the other hand, a recess 13 having a recessed lower surface is formed on the lower surface of the base material 10 to communicate with the through-hole 11.

The electrode 14 is made of, for example, a metal thin film formed on the surface (lower surface) of the base material 10 in which the end of the through-hole 11 on the inlet side is open. Examples of the water absorbing structure 15 include fibers such as cotton and silk, and a porous ceramic substrate. Examples of the electrode 16 include a porous metal thin film formed on the surface of the water absorbing structure 15 by using, for example, a plating technique, a porous metal thin film in which fibers of the water absorbing structure 15 are impregnated with a conductive polymer, and a porous metal thin film in which conductive fibers are woven.

As illustrated in FIG. 8, the wearable sensor 1 is attached to the body of a wearer such that the lower surface of the base material 10 faces the skin 100 of the wearer. The reference numeral 101 in FIG. 8 denotes sweat glands of the wearer.

When the wearer sweats, the sweat 102 is introduced from the inside of the recess 13 of the base material 10 into the through-hole 11 due to capillary action. Due to the increase in an amount of sweat, the sweat 102 rises in the through-hole 11 and reaches the recess 12.

As illustrated in the enlarged view of FIG. 9, a water repellent portion 17 is provided on the inner surface of the recess 12. In a case where a hydrophilic material is used for the base material 10, the water repellent portion 17 may be formed by applying water-repellent surface treatment to the inner surface of the recess 12. When a water-repellent material is used for the base material 10, the water repellent portion 17 may be provided by leaving only the inner surface of the recess 12 as a water-repellent material.

When the sweat 102 reaches the recess 12, the sweat becomes a spherical droplet 102a as illustrated in FIG. 9. As an amount of sweat increases, a diameter of the droplet 102a increases and eventually reaches the electrode 16 and the water absorbing structure 15. The droplet 102a reaching the electrode 16 and the water absorbing structure 15 passes through a large number of holes of the electrode 16 and a large number of holes of the water absorbing structure 15 due to a capillary phenomenon, and evaporates while moving in the water absorbing structure 15. As a result, the droplet 102a disappears. Thus, due to the formation and disappearance of the droplet 102a, energization between the electrodes 14 and 16 of the wearable sensor 1 repeatedly occurs.

In the present embodiment, the structure of the wearable sensor 1 has been described by taking the structure in FIGS. 8 and 9 as an example, but the present invention is not limited thereto. A wearable sensor having another structure may be used as long as a current intermittently flows between electrodes due to droplets intermittently generated in the wearable sensor by a wearer's sweat.

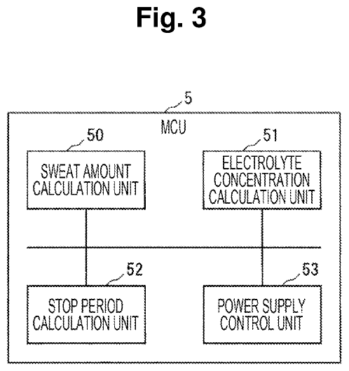

FIG. 3 is a functional block diagram of the MCU 5 of the present embodiment. The MCU 5 functions as a sweat amount calculation unit 50, an electrolyte concentration calculation unit 51, a stop period calculation unit 52, and a power supply control unit 53.

The sweat amount calculation unit 50 calculates an amount of sweat of the wearer on the basis of the energization characteristics between the electrodes of the wearable sensor 1.

The electrolyte concentration calculation unit 51 calculates an electrolyte concentration in the wearer's sweat on the basis of the energization characteristics between the electrodes of the wearable sensor 1.

The power supply control unit 53 stops the supply of power from the power supply unit 7 to the wearable sensor 1, the AFE unit 2, the data recording unit 3, the sweat amount calculation unit 50, the electrolyte concentration calculation unit 51, and the communication unit 6 when the supply-of-power stop condition is satisfied, and restarts the supply of power to the wearable sensor 1, the AFE unit 2, the data recording unit 3, the sweat amount calculation unit 50, the electrolyte concentration calculation unit 51, and the communication unit 6 when the supply-of-power stop period is ended.

The stop period calculation unit 52 calculates the supply-of-power stop period on the basis of the energization characteristics between the electrodes of the wearable sensor 1.

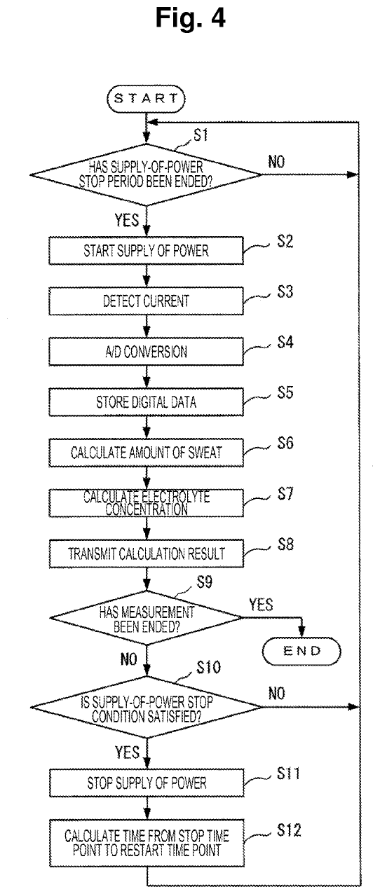

FIG. 4 is a flowchart for describing an operation of the sweating analysis device of the present embodiment. First, the power supply control unit 53 determines whether the supply-of-power stop period determined by the stop period calculation unit 52 has been ended (step S1 in FIG. 4). Details of the supply-of-power stop period will be described later.

In a case where it is determined that the supply-of-power stop period has been ended (YES in step S1), the power supply control unit 53 starts the supply of a power supply voltage from the power supply unit 7 to the AFE unit 2, the data recording unit 3, the sweat amount calculation unit 50, the electrolyte concentration calculation unit 51, and the communication unit 6 (step S2 in FIG. 4). The voltage is applied between the electrodes 14 and 16 of the wearable sensor 1 via the AFE unit 2.

In an initial state, since the supply-of-power stop period is not set, the flow proceeds to step S2 without performing the determination in step S1.

The AFE unit 2 detects a current flowing between the electrodes 14 and 16 of the wearable sensor 1 (step S3 in FIG. 4).

The data recording unit 3 converts a signal detected and amplified by the AFE unit 2 into digital data at a predetermined sampling rate (step S4 in FIG. 4), and stores the digital data in the storage unit 4 (step S5 in FIG. 4). In this case, the data recording unit 3 adds information regarding a sampling time to the digital data and stores the digital data in the storage unit 4. As described above, time series data of the current is stored in the storage unit 4.

The sweat amount calculation unit 50 calculates an amount of sweat Q[L/(cm²·min)] of the wearer of the wearable sensor 1 on the basis of the digital data stored in the storage unit 4 (step S6 in FIG. 4). Specifically, the sweat amount calculation unit 50 can calculate the amount of sweat Q[L/(cm²·min)] by dividing a volume V[L] of the sweat droplet 102a by a cycle t [min] from the immediately preceding current peak to the latest current peak and an area S[cm²] of the wearer's skin 100 covered with the wearable sensor 1.

As described in Patent Literature 1, the volume V of the sweat droplet 102a generated between the electrodes 14 and 16 of the wearable sensor 1 can be obtained in advance as an actual value. The area S of the wearer's skin 100 covered with the wearable sensor 1 is also a known value.

The electrolyte concentration calculation unit 51 calculates an electric resistivity p of sweat that changes depending on an electrolyte concentration CSW in the sweat of the wearer, and calculates the electrolyte concentration CSW in the sweat from the electric resistivity p (step S7 in FIG. 4). Specifically, the electrolyte concentration calculation unit 51 calculates a resistance by dividing the value of the known voltage applied between the electrodes 14 and 16 by the AFE unit 2 by the current peak value at the time of the latest energization indicated by the digital data stored in the storage unit 4. The electrolyte concentration calculation unit 51 calculates the electric resistivity p on the basis of the resistance, the known distance between the electrodes 14 and 16, and the cross-sectional area of sweat between the electrodes 14 and 16. As the cross-sectional area of sweat, a specified value when the cross-sectional area of sweat between the electrodes 14 and 16 is assumed to be constant may be used.

It is known that there is a linear relationship between the electric resistivity p of sweat and the electrolyte concentration CSW (mainly the concentration of NaCl) in sweat. The electrolyte concentration calculation unit 51 calculates the electrolyte concentration CSW from the electric resistivity p on the basis of the known relationship between the electric resistivity p and the electrolyte concentration CSW.

The communication unit 6 transmits the calculation result in the sweat amount calculation unit 50 and the calculation result in the electrolyte concentration calculation unit 51 to an external device (not illustrated) such as a smartphone (step S8 in FIG. 4).

Next, the power supply control unit 53 determines whether the supply-of-power stop condition is satisfied (step S10 in FIG. 4). In the present embodiment, the time from a time point at which the current starts flowing between the electrodes 14 and 16 of the wearable sensor 1 to a time point at which the supply-of-power stop condition is satisfied is defined as t₁. In the example in FIG. 1, the time from A1 to B1 and the time from A2 to B2 are t₁. However, a length of t₁ may change for each current peak. As a method of determining the supply-of-power stop condition, for example, there are the following two methods.

### First Determination Method

In a first determination method, it is assumed that a time point t₃ required from the start of a current flow between the electrodes 14 and 16 to the occurrence of a current peak is known in advance. In this case, the power supply control unit 53 may determine that the supply-of-power stop condition is satisfied when a certain period of time has elapsed from the latest time point at which the current starts flowing between the electrodes 14 and 16. The fixed time at this time is t₁, and is set in advance as a value equal to or longer than time point t₃. Second determination method

In a second determination method, a length of the time point t₁ may change for each current peak. Specifically, the power supply control unit 53 determines that the supply-of-power stop condition is satisfied when a current peak occurs after the supply of a power supply voltage is started in the immediately preceding step S2. In this case, the time from a time point at which the current starts flowing between the electrodes 14 and 16 to a time point at which the current peak occurs is t₁.

When it is determined that the supply-of-power stop condition is satisfied (YES in step S10), the power supply control unit 53 stops the supply of the power supply voltage from the power supply unit 7 to the AFE unit 2, the data recording unit 3, the sweat amount calculation unit 50, the electrolyte concentration calculation unit 51, and the communication unit 6 (step S11 in FIG. 4). When the supply of the power supply voltage to the AFE unit 2 is stopped, the application of the voltage between the electrodes 14 and 16 is also stopped.

Next, the stop period calculation unit 52 calculates a time point t₂ from the supply-of-power stop time point to the supply-of-power restart time point (step S12 in FIG. 4). In the example in FIG. 1, for example, a supply-of-power stop time point is B1, and a supply-of-power restart time point is C1. As a method of calculating the time point t₂ (supply-of-power stop period), for example, there are the following three methods.

### First Calculation Method

As described above, the time from the latest time point at which the current starts flowing between the electrodes 14 and 16 to the supply-of-power stop time point at which the supply-of-power stop condition is satisfied in step S10 is defined as t₁[sec]. A cycle of the current peak corresponding to the maximum amount of sweat Qₘₐₓ of the wearer is defined as tₘᵢₙ[sec]. The stop period calculation unit 52 calculates the time point t₂[sec] to satisfy the following Formula (1).

$$0 < {t}_{2} < {t}_{min} - {t}_{1} \qquad ( 1 )$$

As described above, since an amount of sweat is calculated from a volume of sweat droplets, a current peak cycle, and an area of the wearer's skin covered by the wearable sensor 1, the current peak cycle tₘᵢₙ corresponding to the maximum amount of sweat Qₘₐₓ can be obtained in advance. As the maximum amount of sweat Qₘₐₓ, a known value obtained in past measurement may be used. The stop period calculation unit 52 may set the longest time within a range satisfying the condition of the Formula (1) as t₂.

### Second Calculation Method

The latest amount of sweat calculated by the sweat amount calculation unit 50 in step S6 is denoted by Q, and a cycle of the current peak corresponding to the amount of sweat Q is denoted by t[sec]. The current peak cycle t[sec] is a value used in the calculation of the amount of sweat Q in step S6. The stop period calculation unit 52 calculates the time point t₂[sec] so as to satisfy the following Formula (2).

$$0 < {t}_{2} < t - {t}_{1} \qquad ( 2 )$$

Similarly to the case of Formula (1), the stop period calculation unit 52 may set the longest time within a range satisfying the condition of Formula (2) as t₂.

### Third Calculation Method

As described above, the latest amount of sweat calculated by the sweat amount calculation unit 50 is Q, and the known maximum amount of sweat of the wearer is Qₘₐₓ. When the sum Q+ΔQ of the latest amount of sweat Q and a predetermined value ΔQ is smaller than the maximum amount of sweat Qₘₐₓ (Q+ΔQ<Qₘₐₓ), the stop period calculation unit 52 calculates a cycle t*[sec] corresponding to the amount of sweat Q+ΔQ of the current peak and calculates the time point t₂[sec] to satisfy the following Formula (3).

$$0 < {t}_{2} < {t}^{⋆} - {t}_{1} \qquad ( 3 )$$

The predetermined value ΔQ(ΔQ>o) is a parameter related to an increase rate of an amount of sweat of the wearer, and may be set in advance to an actual value from a result obtained in past measurement. Similarly to the case of Formula (1), the stop period calculation unit 52 may set the longest time within a range satisfying the condition of Formula (3) as t₂.

When the sum Q+ΔQ of the latest amount of sweat Q and the predetermined value ΔQ is equal to or more than the maximum amount of sweat Qₘₐₓ (Q+ΔQ≥Qₘₐₓ), the stop period calculation unit 52 calculates the time point t₂[sec] to satisfy the above Formula (1).

For example, the sweating analysis device repeatedly executes the processes in steps S1 to S8 and S10 to S12 until there is an instruction to end the measurement from the wearer (YES in step S9 in FIG. 4).

In the determination in the next step S1, when an elapsed time from the supply-of-power stop time point at which the supply-of-power stop condition is satisfied in the immediately preceding step S10 is less than t₂ calculated by the stop period calculation unit 52, the power supply control unit 53 stands by in a supply-of-power stop state. The power supply control unit 53 determines that the supply-of-power stop period is ended when an elapsed time from the supply-of-power stop time point is t₂ or more.

As described above, according to the present embodiment, since the power supply voltage is supplied to the wearable sensor 1, the AFE unit 2, the data recording unit 3, the sweat amount calculation unit 50, the electrolyte concentration calculation unit 51, and the communication unit 6 only for the time required for calculating an amount of sweat and an electrolyte concentration, it is possible to perform the measurement of biological information such as an amount of sweat and an electrolyte concentration while further saving power.

### Second Embodiment

Next, a second embodiment of the present invention will be described. In the present embodiment, the wearable sensor 1 disclosed in WO 2021/038758 is used.

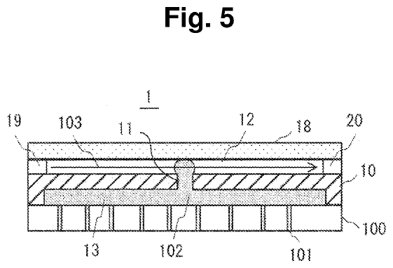

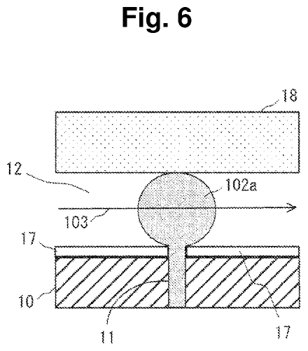

FIG. 5 is a sectional view of the wearable sensor 1 of the present embodiment, and FIG. 6 is an enlarged view of FIG. 5. The wearable sensor 1 includes a base material 10 having a through-hole 11 serving as a liquid flow path and a recess 12 communicating with the end of the through-hole 11 on an outlet side, a water absorbing structure 18 disposed on a surface (upper surface) of the base material 10 on the outlet side to be in contact with a liquid flowing out from an opening of the through-hole 11 on the outlet side to the recess 12, a laser diode (LD) 19 disposed in the recess 12 and emitting light to a path in the recess 12 passing through a position on the opening of the through-hole 11 on the outlet side along the surface of the base material 10 on the outlet side, and a photodiode (PD) 20 disposed in the recess 12 to face the LD 19 across the position on the opening of the through-hole 11 on the outlet side and receiving light from the LD 19.

Examples of the water absorbing structure 18 include fibers such as cotton and silk, and a porous ceramic substrate. The water absorbing structure 18 does not need to cover the whole surface of the recess 12 and the opening of the through-hole 11 on the outlet side, but may be disposed to be able to contact a droplet flowing out from the opening of the through-hole 11 on the outlet side to the recess 12.

As illustrated in FIG. 5, the wearable sensor 1 is attached to the wearer's body such that the lower surface of the base material 10 faces the wearer's skin 100.

When the wearer sweats, the sweat 102 is introduced from the inside of the recess 13 of the base material 10 into the through-hole 11 due to capillary action. Due to the increase in an amount of sweat, the sweat 102 rises in the through-hole 11 and reaches the recess 12 provided on the upper surface of base material 10 to communicate with the through-hole 11 (FIG. 6).

As in the first embodiment, the water repellent portion 17 is provided on the inner surface of the recess 12. When the sweat 102 reaches the recess 12, the sweat 102 becomes a spherical droplet 102a as illustrated in FIG. 6. As an amount of sweat increases, a diameter of the droplet 102a increases and eventually reaches the water absorbing structure 18. The droplet 102a reaching the water absorbing structure 18 passes through a large number of holes of the water absorbing structure 18 due to a capillary phenomenon, and evaporates while moving in the water absorbing structure 18. As a result, the droplet 102a disappears.

As illustrated in FIGS. 5 and 6, the LD 19 that is a light emitting element emits light to a path in the recess 12 passing through a position on the opening of the through-hole 11 on the outlet side along the surface (upper surface) of the base material 10 on the outlet side at the time of measuring an amount of sweat.

The PD 20 that is a light receiving element receives light from the LD 19.

When the droplet 102a of the sweat 102 is formed, light 103 emitted from the LD 19 propagates through the respective media in the order of the air in the recess 12, the droplet 102a, and the air in the recess 12, and enters the PD 20. When the droplet 102a disappears, the light 103 propagates in the air in the recess 12 and enters the PD 20. When the droplet 102a is formed again, the light 103 propagates through the respective media in the order of the air in the recess 12, the droplet 102a, and the air in the recess 12, and enters the PD 20. As described above, a difference in the medium through which the light 103 propagates is reflected in an amount of light received by the PD 20. That is, a photocurrent flowing through the PD 20 changes due to the formation and disappearance of the droplet 102a.

The configurations of the AFE unit 2, the data recording unit 3, the storage unit 4, the MCU 5, the communication unit 6, and the power supply unit 7 of the sweating analysis device are similar to those in the first embodiment.

The AFE unit 2 may amplify an output signal (photocurrent) of the PD 20.

In the present embodiment, when the droplet 102a of the sweat 102 is formed, the photocurrent of the PD 20 decreases, and when the droplet 102a disappears, the photocurrent of the PD 20 increases.

Therefore, instead of a time point at which a current starts flowing between the electrodes 14 and 16 of the first embodiment, a time point at which a change starts to appear in the photocurrent of the PD 20 due to the droplet 102a may be employed.

As described above, when the droplet 102a is formed, a photocurrent of the PD 20 decreases to be equal to or less than a current threshold value. Conversely, when the droplet 102a disappears, the photocurrent of the PD 20 increases and exceeds the current threshold value. The stop period calculation unit 52 and the power supply control unit 53 may set a time point at which the photocurrent of the PD 20 exceeds the current threshold value and becomes equal to or less than the current threshold value as a time point at which a change starts appearing in the photocurrent (a time point at which the current starts flowing in the first embodiment).

In the first embodiment, an upward peak of the current between the electrodes 14 and 16 is detected, and it is determined whether or not the supply-of-power stop condition is satisfied, and the supply-of-power stop period t2, the amount of sweat Q, and the electrolyte concentration CSW are calculated. On the other hand, in the present embodiment, a downward peak of the photocurrent of the PD 20 may be detected, and it is determined whether or not the supply-of-power stop condition is satisfied, and the supply-of-power stop period t2, the amount of sweat Q, and the electrolyte concentration CSW may be calculated.

In the present embodiment, the power supply control unit 53 may start supplying a power supply voltage from the power supply unit 7 to the LD 19, the PD 20, the AFE unit 2, the data recording unit 3, the sweat amount calculation unit 50, the electrolyte concentration calculation unit 51, and the communication unit 6 of the wearable sensor 1 in step S2, and may stop supplying the power supply voltage from the power supply unit 7 to the LD 19, the PD 20, the AFE unit 2, the data recording unit 3, the sweat amount calculation unit 50, the electrolyte concentration calculation unit 51, and the communication unit 6 in step S11. The other constituent s are the same as those in the first embodiment.

In the first and second embodiments, an example has been described in which an electrical signal derived from an amount of sweat and an electrolyte concentration of a user is detected by a wearable sensor as disclosed in Patent Literature 1 and WO 2021/038758, and timings of stopping and restarting the supply of power are controlled on the basis of time-series data of the electrical signal.

However, the present invention is also applicable to a case where a wearable sensor measures biological information having periodicity such as an electrocardiogram waveform, a respiratory motion, and a pulse wave of a user, and calculates a feature amount such as a heart rate, a respiratory rate, and a pulse rate.

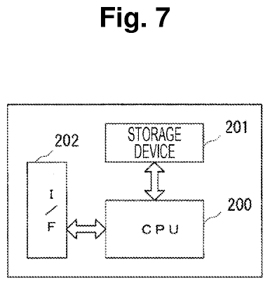

The data recording unit 3, the storage unit 4, the MCU 5, and the communication unit 6 described in the first and second embodiments can be realized by a computer including a central processing unit (CPU), a storage device, and an interface, and a program for controlling these hardware resources. A configuration example of this computer is illustrated in FIG. 7. The computer includes a CPU 200, a storage device 201, and an interface device (I/F) 202. The hardware such as an ADC of the data recording unit 3, the hardware of the communication unit 6, the power supply unit 7, and the like are connected to the I/F 202. In such a computer, a program for realizing the sweating analysis method of embodiments of the present invention is stored in the storage device 201. The CPU 200 executes the processing described in the first and second embodiments according to the program stored in the storage device 201.

### INDUSTRIAL APPLICABILITY

Embodiments of the present invention can be applied to a technique for analyzing an amount of sweat and an electrolyte concentration in sweat.

Reference Signs List

| | |
|---|---|
| 1 | Wearable sensor |
| 2 | AFE unit |
| 3 | Data storage unit |
| 4 | Storage unit |
| 5 | MCU |
| 6 | Communication unit |
| 7 | Power supply unit |
| 10 | Base material |
| 11 | Through-hole |
| 12, 13 | Recess |
| 14, 16 | Electrode |
| 15 | Water absorbing structure |
| 17 | Water repellent portion |
| 18 | Water absorbing structure |
| 19 | Laser diode |
| 20 | Photodiode |
| 50 | Sweat amount calculation unit |
| 51 | Electrolyte concentration calculation unit |
| 52 | Stop period calculation unit |
| 53 | Power supply control unit |

1-8. (canceled)

9. A sweating analysis device comprising:
a wearable sensor configured to output an electrical signal derived from an amount of sweat and an electrolyte concentration in the sweat, the sweat being secreted from skin of a wearer of the wearable sensor;
a storage device comprising instructions; and
one or more processors in communication with the storage device, wherein the one or more processors execute the instructions to:
calculate the amount of sweat of the wearer based on the electrical signal obtained by the wearable sensor;
calculate the electrolyte concentration in the sweat of the wearer based on the electrical signal obtained by the wearable sensor;
a power supply configured to supply power to the wearable sensor and the one or more processors; and
a power supply controller configured to stop supplying the power from the power supply to the wearable sensor and the one or more processors when a supply-of-power stop condition is satisfied, and restart supplying the power to the wearable sensor and the one or more processors when a supply-of-power stop period is ended, wherein the one or more processors execute the instructions to further:
calculate the supply-of-power stop period based on the electrical signal obtained by the wearable sensor.

10. The sweating analysis device according to claim 9, wherein:
the electrical signal is a current that is changed by a droplet intermittently generated in the wearable sensor due to sweating of the wearer; and
the power supply controller is configured to determine that the supply-of-power stop condition is satisfied when a certain period of time has elapsed from a time point at which a change in the current of the electrical signal starts due to the droplet.

11. The sweating analysis device according to claim 9, wherein:
the electrical signal is a current that is changed by a droplet intermittently generated in the wearable sensor due to sweating of the wearer; and
the power supply controller is configured to determine that the supply-of-power stop condition is satisfied when a peak of the current occurs after supply of the power to the wearable sensor and the one or more processors is started.

12. The sweating analysis device according to claim 11, wherein:
the electrical signal is a current that is changed by a droplet intermittently generated in the wearable sensor due to sweating of the wearer; and
when t₁ is a time from a time point at which a change in the current starts due to the droplet to a supply-of-power stop time point at which the supply-of-power stop condition is satisfied, tₘᵢₙ is a cycle of the peak occurring in the current and corresponding to a known maximum amount of sweat of the wearer, and t₂ is the supply-of-power stop period, the one or more processors execute the instructions to calculate the supply-of-power stop period t₂ to satisfy 0<t₂<tₘᵢₙ−t₁.

13. The sweating analysis device according to claim 9, wherein:
the electrical signal is a current that is changed by a droplet intermittently generated in the wearable sensor due to sweating of the wearer; and
when t₁ is a time from a time point at which a change in the current starts appearing due to the droplet to a supply-of-power stop time point at which the supply-of-power stop condition is satisfied, Q is the amount of sweat, t is a cycle of a peak occurring in the current and corresponding to the amount of sweat Q, and t₂ is the supply-of-power stop period, the one or more processors execute the instructions to calculate the supply-of-power stop period t₂ to satisfy 0<t₂<t−t₁.

14. The sweating analysis device according to claim 9 wherein:
the electrical signal is a current that is changed by a droplet intermittently generated in the wearable sensor due to sweating of the wearer; and
when t₁ is a time from a time point at which a change in the current starts appearing due to the droplet to a supply-of-power stop time point at which the supply-of-power stop condition is satisfied, Q is the amount of sweat, Qₘₐₓ is a known maximum amount of sweat of the wearer, tₘᵢₙ is a cycle of a peak occurring in the current and corresponding to the maximum amount of sweat Qₘₐₓ, and t₂ is the supply-of-power stop period, the one or more processors execute the instructions to:
calculate a cycle t* of the peak occurring in the current and corresponding to an amount of sweat Q+ΔQ and calculate the supply-of-power stop period t₂ to satisfy 0<t₂<t*−t₁ in a case where a sum Q+ΔQ of the amount of sweat Q and a predetermined value ΔQ is less than the maximum amount of sweat Qₘₐₓ; and
calculate the supply-of-power stop period t₂ to satisfy 0<t₂<tₘᵢₙ−t₁ in a case where the sum Q+ΔQ of the amount of sweat Q and the predetermined value ΔQ is equal to or more than the maximum amount of sweat Qₘₐₓ.

15. The sweating analysis device according to claim 9, wherein the electrical signal is a current that is changed by a droplet intermittently generated in the wearable sensor due to sweating of the wearer;
the one or more processors execute the instructions to calculate the amount of sweat of the wearer based a cycle of a peak of the current; and
the one or more processors execute the instructions to calculate the electrolyte concentration in the sweat of the wearer based on a value of the peak of the current.

16. A sweating analysis method comprising:
detecting, by a wearable sensor, an electrical signal derived from an amount of sweat and an electrolyte concentration in the sweat, the sweat being secreted from skin of a wearer of the wearable sensor;
calculating the amount of sweat of the wearer based on the electrical signal obtained by the wearable sensor;
calculating the electrolyte concentration in the sweat of the wearer based on the electrical signal obtained by the wearable sensor;
stopping supply of power from a power supply to the wearable sensor when a supply-of-power stop condition is satisfied;
calculating a supply-of-power stop period based on the electrical signal obtained by the wearable sensor; and
restarting the supply of power to the wearable sensor when the supply-of-power stop period is ended.

17. The sweating analysis method according to claim 16, wherein:
the electrical signal is a current that is changed by a droplet intermittently generated in the wearable sensor due to sweating of the wearer; and
the method further comprises determining the supply-of-power stop condition is satisfied when a certain period of time has elapsed from a time point at which a change in the current of the electrical signal starts due to the droplet.

18. The sweating analysis method according to claim 16, wherein:
the electrical signal is a current that is changed by a droplet intermittently generated in the wearable sensor due to sweating of the wearer; and
the method further comprises determining that the supply-of-power stop condition is satisfied when a peak of the current occurs after supply of the power to the wearable sensor is started.

19. The sweating analysis method according to claim 18, wherein:
the electrical signal is a current that is changed by a droplet intermittently generated in the wearable sensor due to sweating of the wearer; and
when t₁ is a time from a time point at which a change in the current starts due to the droplet to a supply-of-power stop time point at which the supply-of-power stop condition is satisfied, tₘᵢₙ is a cycle of the peak occurring in the current and corresponding to a known maximum amount of sweat of the wearer, and t₂ is the supply-of-power stop period, calculating the supply-of-power stop period comprises calculating the supply-of-power stop period t₂ to satisfy 0<t₂<tₘᵢₙ−t₁.

20. The sweating analysis method according to claim 16, wherein:
the electrical signal is a current that is changed by a droplet intermittently generated in the wearable sensor due to sweating of the wearer; and
when t₁ is a time from a time point at which a change in the current starts appearing due to the droplet to a supply-of-power stop time point at which the supply-of-power stop condition is satisfied, Q is the amount of sweat, t is a cycle of a peak occurring in the current and corresponding to the amount of sweat Q, and t₂ is the supply-of-power stop period, calculating the supply-of-power stop period comprises calculating the supply-of-power stop period t₂ to satisfy 0<t₂<t−t₁.

21. The sweating analysis method according to claim 16, wherein:
the electrical signal is a current that is changed by a droplet intermittently generated in the wearable sensor due to sweating of the wearer; and
when t₁ is a time from a time point at which a change in the current starts appearing due to the droplet to a supply-of-power stop time point at which the supply-of-power stop condition is satisfied, Q is the amount of sweat, Qₘₐₓ is a known maximum amount of sweat of the wearer, tₘᵢₙ is a cycle of a peak occurring in the current and corresponding to the maximum amount of sweat Qₘₐₓ, and t₂ is the supply-of-power stop period, calculating the supply-of-power stop period comprises:
calculating a cycle t* of the peak occurring in the current and corresponding to an amount of sweat Q+ΔQ and calculate the supply-of-power stop period t₂ to satisfy 0<t₂<t*−t₁ in a case where a sum Q+ΔQ of the amount of sweat Q and a predetermined value ΔQ is less than the maximum amount of sweat Qₘₐₓ; and
calculating the supply-of-power stop period t₂ to satisfy 0<t₂<tₘᵢₙ−t₁ in a case where the sum Q+ΔQ of the amount of sweat Q and the predetermined value ΔQ is equal to or more than the maximum amount of sweat Qₘₐₓ.

22. The sweating analysis method according to claim 16, wherein
the electrical signal is a current that is changed by a droplet intermittently generated in the wearable sensor due to sweating of the wearer;
calculating the amount of sweat comprises calculating the amount of sweat of the wearer based a cycle of a peak of the current; and
calculating the amount of sweat comprises calculating the electrolyte concentration in the sweat of the wearer based on a value of the peak of the current.
